(19) United States
(12) Patent Application Publication
(10) Pub. No.: US 2004/0263779 A1
(43) Pub. Date: Dec. 30, 2004

(54) Hartmann-Shack wavefront measurement

(75) Inventor: Russell Schroder, San Jose, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: VISX, Inc., Santa Clara, CA

(21) Appl. No.: 10/462,102

(22) Filed: Jun. 12, 2003

(51) Int. Cl. A61B 3/10

(52) U.S. Cl. 351/205

(57) ABSTRACT

The present invention provides systems and methods for imaging, diagnosing and treating an eye of a patient. In one embodiment, a system (100) includes an image source arranged to direct an image posteriorly through the eye optical tissues and onto the retina, and a wavefront sensor (140). The wavefront sensor, comprising a lenslet array (170) and a CMOS imaging device, is oriented to sense the image transmitted anteriorly by the optical tissue. A processor may be coupled to the wavefront sensor for processing the image. In some embodiments, the CMOS imaging device comprises a CMOS camera having a dynamic range that is at least about 100 db, to provide the ability to image high contrasting images, such as a series of bright lights on a dark background as may be produced when imaging the eye.

### BACKGROUND OF THE INVENTION

The present invention relates generally to wavefront measurement devices, systems and methods. In particular, the present invention relates to improved Hartmann-Shack wavefront measurement devices, systems and methods to more accurately measure optical aberrations in a patient's eye.

Laser eye surgical systems can be used for a variety of procedures, including ablation procedures to remove targeted stroma of the cornea to change the comea's contour. Such a technique may be useful, for example, for correcting myopia, hyperopia, astigmatism, and the like. Laser eye surgical systems typically employ a system that can track and measure the optical characteristics of the patient's eye. For example, Zernike polynomials have been employed to model the optical surface. Once the eye profile is determined, a variety of algorithms may be used to calculate a pattern of laser pulses used to reshape the cornea to perform the desired correction. As one would imagine, an accurate mapping of the eye profile is crucial to the success of the surgical procedure.

One promising eye measurement system uses wavefront sensor data in combination with mathematical modeling to map the optical surface of the eye. A wavefront measurement of the eye creates a high order aberration map that permits the assessment of aberrations throughout the optical pathway of the eye, e.g., both internal aberrations and aberrations on the corneal surface. The aberration information can then be used to compute a custom ablation pattern so that the surgical laser system can correct the aberrations of the patient's eye. Although such wavefront measurements are often described in the context of laser surgical systems, such measurements may also be used to formulate refractive correction patterns in alternative eye treatment procedures and systems such as for use in radial keratotomy, intraocular lenses, corneal ring implants, and the like.

One exemplary wavefront measurement system is the VISX WaveScan® System, which uses Hartmann-Shack wavefront sensors that can quantify aberrations throughout the entire optical system, including first and second-order sphero-cylindrical errors, coma, and third through sixth order aberrations related to coma, astigmatism, and spherical aberrations. The aberrations can be displayed to the surgeon in the form of an AcuityMap® and/or an aberration map, for example. The wavefront aberrations measured with a wavefront sensor provide a map of optical aberration across the pupil of the eye. The aberration map can then be used to plan a refractive correction for improving vision quality via wavefront-guided laser vision correction or other vision correction means.

The Hartmann-Shack system generates a spot pattern that is used to produce the eye profile using a set of algorithms. Notwithstanding the success of this system, still further improvements would be desirable. For example, the Hartmann-Shack pattern is an array of very bright spots on a dark background. The CCD imaging camera has limitations in the processing of these high contrast images. If the contrast is too great, adjacent pixels bleed into one another in a process called “blooming,” resulting in a loss of image data for those pixels. Loss of image data negatively affects the ability to accurately map the eye profile.

In light of the above, it would be desirable to provide improved optical measurement techniques, particularly for use in the measurement of the eye for refractive correction purposes.

### BRIEF SUMMARY OF THE INVENTION

The present invention provides apparatus, systems and methods for measuring wavefronts using an optical wavefront imaging system, such as the VISX WaveScan Wavefront® System. It should be appreciated however, that the present invention can be used in or in conjunction with a wide range of eye measurement devices, such as those manufactured and/or sold by Bausch & Lomb, Wavefront Sciences, Alcon Laboratories, and the like. The present invention will also find use in a large variety of wavefront imaging systems, and are not limited to eye measurement devices.

The present invention provides systems for diagnosing an eye of a patient, the eye having a retina and optical tissues. In one embodiment the system includes an image source arranged to direct an image posteriorly through the optical tissues and onto the retina, and a wavefront sensor. The wavefront sensor, comprising a lenslet array and a CMOS imaging device, is oriented to sense the image as transmitted anteriorly by the optical tissue. The system further includes a processor coupled to the wavefront sensor for processing the image. In alternative embodiments, the CMOS imaging device comprises a CMOS camera having a dynamic range that is at least about 100 db, that is at least about 120 db, or the like. In some embodiments, the wavefront sensor comprises a Hartmann-Shack sensor. In this manner, the CMOS imaging device provides the ability to image high contrasting images, such as a series of bright lights on a dark background as may be produced when imaging the eye.

In one aspect, the processor is adapted for identifying an aberration of the eye based at least in part on the processed image. In this manner, images received and processed are available to help assist physicians or users diagnose a patient's eye and plan appropriate treatments, such as vision correction procedures.

In one aspect, the CMOS imaging device includes a plurality of pixels, including a first pixel. The imaging device is adapted to prevent a blooming effect between the first pixel and at least some of the adjacent pixels. In this manner, data from the adjoining pixels will not bleed together or be otherwise lost.

In another embodiment, an apparatus of the present invention is adapted for measuring optical aberrations of an eye. The apparatus includes a light source arranged to direct a light beam along an optical axis into the optical tissues and onto the retina, and a wavefront sensor. The wavefront sensor, which includes a lenslet array and a CMOS imaging device, is oriented to sense a returned image from the optical tissue. Further, the CMOS imaging device has a dynamic range that is at least about 100 db. In this manner, the CMOS imaging device has exemplary anti-blooming characteristics based in part on the increased dynamic range of the CMOS device pixels.

In one aspect, the wavefront sensor is coupled to a processor adapted for processing the returned image and determining a treatment profile for the eye. In a particular aspect, the processor is coupled to a laser adapted for generating a photoablative laser beam suitable for removal of corneal tissue of the eye so as to correct refraction.

The present invention further provides methods for treating a patient's eye, for generating a profile of a patient's eye, and the like. In one such embodiment, the method includes projecting a light into the patient's eye and onto a retina, receiving a wavefront profile from the eye with an imaging device having a dynamic range that is at least about 100 db, and calculating a tomographic wavefront error map of the eye to identify at least one aberration of the eye.

In one aspect, the method further includes corresponding the aberration with a tissue structure of the eye for a subsequent treatment. The method may further include ablating a portion of the patient's eye in a defined pattern, the defined pattern based at least in part on the tomographic wavefront error map.

A further understanding of the nature and advantages of the invention will become apparent by reference to the remaining portions of the specification and drawings

### BRIEF DESCRIPTION OF THE DRAWINGS

FIG. 1A illustrates light reflected off a point on the retina in which the eye has no aberrations;

FIG. 1B illustrates light that is reflected off a point on the retina in which the eye has aberrations;

FIG. 2A is a wavefront spot pattern of the eye of FIG. 1A;

FIG. 2B is a distorted wavefront spot pattern from the eye with aberrations of FIG. 1B;

FIG. 3 schematically illustrates a method and system for directly determining a corneal ablation treatment prescription or program from wavefront sensor data;

FIG. 4 schematically illustrates a method and system according to the present invention for imaging a patient's eye; and

FIG. 5 is a simplified flow diagram illustrating a method of imaging an eye according to the present invention.

### DETAILED DESCRIPTION OF THE INVENTION

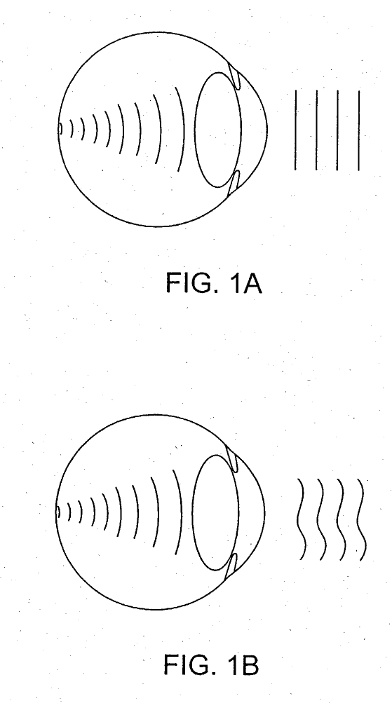

Wavefront systems collect and analyze light that is reflected off of the retina to determine the low order and high order aberrations (if any) that are present in the optical path of the patient's eye. As illustrated in FIG. 1A, light will generally focus to a point in spherical waves through an eye that has no aberrations. However, as shown in FIG. 1B, light will distort when it passes through a refractive medium that has aberrations, such as an irregular cornea or lens. Wavefront sensors, such as Hartmann-Shack sensors, are capable of measuring the distortions in the wavefront as it exits the optical tissue of the eye.

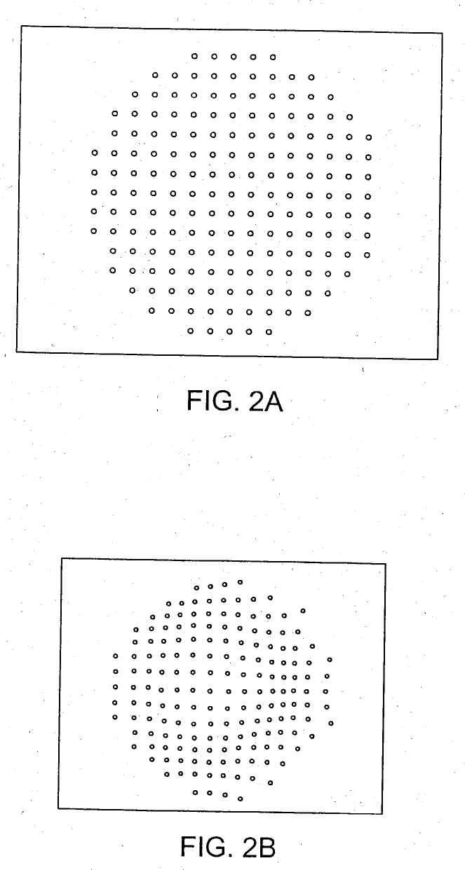

Wavefront systems can segment each wavefront using a series of sub-apertures and focus the light that travels through each sub-aperture onto an imaging device, such as a charge coupled device (CCD), using a series of lenslets corresponding to the sub-apertures. In a flat wavefront, the focal points are in line with the optical axes of the lenslets, and, as shown in FIG. 2A, the resultant spot pattern matches the pattern of the sub-apertures (in this illustration the spot pattern is equidistant.)

When the wavefront is distorted due to aberrations in the eye, each focal point will shift proportionate to the gradient of that part of the wave that passes through the corresponding lenslet. As shown in FIG. 2B, the resultant pattern will have an irregular form.

The wavefront data can be constructed into a color representation of visual acuity or wavefront variations over the entire surface area of the pupil. The map can precisely represent variations in refractive status encompassing the entire optical system, based on measurements taken of the wavefront as it exits the eye. Low order, higher-order, and sphero-cylindrical aberrations can be captured by wavefront systems, such as the VISX WaveScan® System so as to allow the surgeon to make an objective assessment of the wavefront-based refraction. Additional details on imaging corneal profiles may be found in U.S. Pat. Nos. 6,315,413 and 6,419,671 and 6,520,958, and in International Publication WO 02/46801, assigned to the assignee of the present invention and incorporated herein by reference for all purposes.

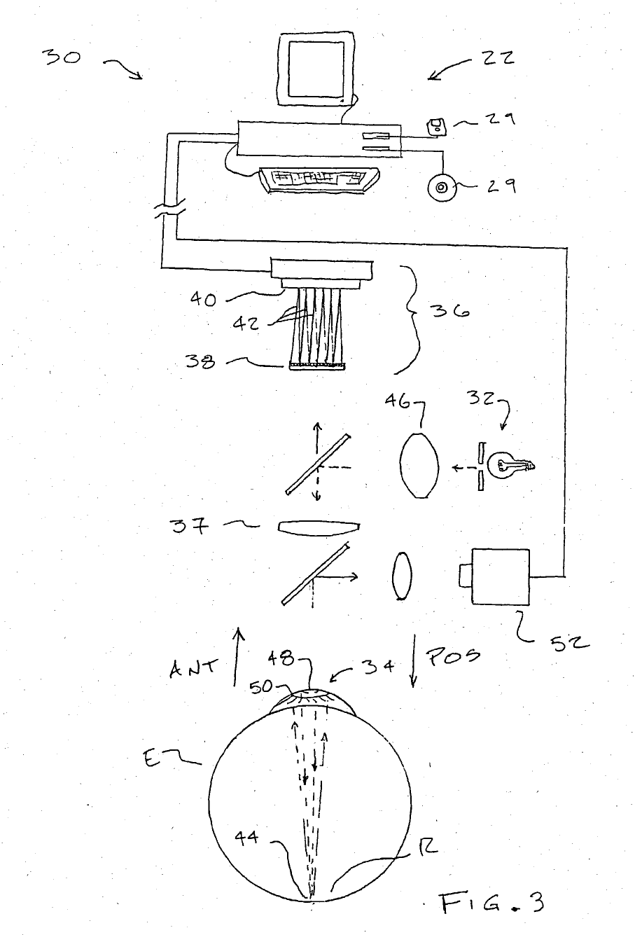

Referring now to FIG. 3, one embodiment of a wavefront sensor system 30 is schematically illustrated in simplified form. In very general terms, wavefront system 30 includes an image source 32 which projects a source image through optical tissues 34 of eye E and so as to form an image 44 upon a surface of retina R. The image from retina R is transmitted by the optical system of the eye (specifically, optical tissues 34), through one or more lens 37 as needed, and imaged onto a wavefront sensor 36 by system optics 38. The wavefront sensor 36 communicates signals to computer 22 for determination of a corneal ablation treatment program. Computer 22 may be the same computer which is used to direct operation of the laser surgery system 10, or at least some or all of the computer components of the wavefront sensor system and laser surgery system may be separate. Data from wavefront sensor 36 may be transmitted to a separate laser system computer via tangible media 29, via an I/O port, via an networking connection such as an intranet or the Internet, or the like.

Wavefront sensor 36 generally comprises a lenslet array 38 and an image sensor 40. As the image from retina R is transmitted through optical tissues 34 and lenslet array 38, the lenslet array separates the transmitted image into an array of beamlets 42, and (in combination with other optical components of the system) images the separated beamlets on the surface of sensor 40. In one embodiment, sensor 40 comprises a charged couple device (CCD). Sensor 40 senses the characteristics of beamlets 42, which can be used to determine the characteristics of an associated region of optical tissues 34. In particular, where image 44 comprises a point or small spot of light, a location of the transmitted spot as imaged by a beamlet can directly indicate a local gradient of the associated region of optical tissue.

Eye E generally defines an anterior orientation (ANT) and a posterior orientation (POS). Image source 32 generally projects an image in a posterior orientation through optical tissues 34 onto retina R. Optical tissues 34 again transmit image 44 from the retina anteriorly toward wavefront sensor 36. Image 44 actually formed on retina R may be distorted by any imperfections in the eye's optical system when the image source is originally transmitted by optical tissues 34. Optionally, image source projection optics 46 may be configured or adapted to decrease any distortion of image 44.

In some embodiments, image source optics may decrease lower order optical errors by compensating for spherical and/or cylindrical errors of optical tissues 34. Higher order optical errors of the optical tissues may also be compensated through the use of an adaptive optic element, such as a deformable mirror. Use of an image source 32 selected to define a point or small spot at image 44 upon retina R may facilitate the analysis of the data provided by wavefront sensor 36. Distortion of image 44 may be limited by transmitting a source image through a central region 48 of optical tissues 34 which is smaller than a pupil 50, as the central portion of the pupil may be less prone to optical errors than the peripheral portion. Regardless of the particular image source structure, it will be generally be beneficial to have well-defined and accurately formed image 44 on retina R.

While reference to sensing of an image 44 is described, it should be understood that a series of wavefront sensor data readings may be taken. For example, a time series of wavefront data readings may help to provide a more accurate overall determination of the ocular tissue aberrations. As the ocular tissues can vary in shape over a brief period of time, a plurality of temporally separated wavefront sensor measurements can avoid relying on a single snapshot of the optical characteristics as the basis for a refractive correcting procedure. Still further alternatives are also available, including taking wavefront sensor data of the eye with the eye in differing configurations, positions, and/or orientations. For example, a patient will often help maintain alignment of the eye with wavefront sensor system 30 by focusing on a fixation target, as described in U.S. Pat. No. 6,004,313, the full disclosure of which is incorporated herein by reference. By varying a focal position of the fixation target as described in that reference, optical characteristics of the eye may be determined while the eye accommodates or adapts to image a field of view at a varying distance. Further alternatives include rotating of the eye by providing alternative and/or moving fixation targets within wavefront sensor system 30.

The location of the optical axis of the eye may be verified by reference to the data provided from a pupil camera 52. In the exemplary embodiment, a pupil camera 52 images pupil 50 so as to determine a position of the pupil for registration of the wavefront sensor data relative to the optical tissues.

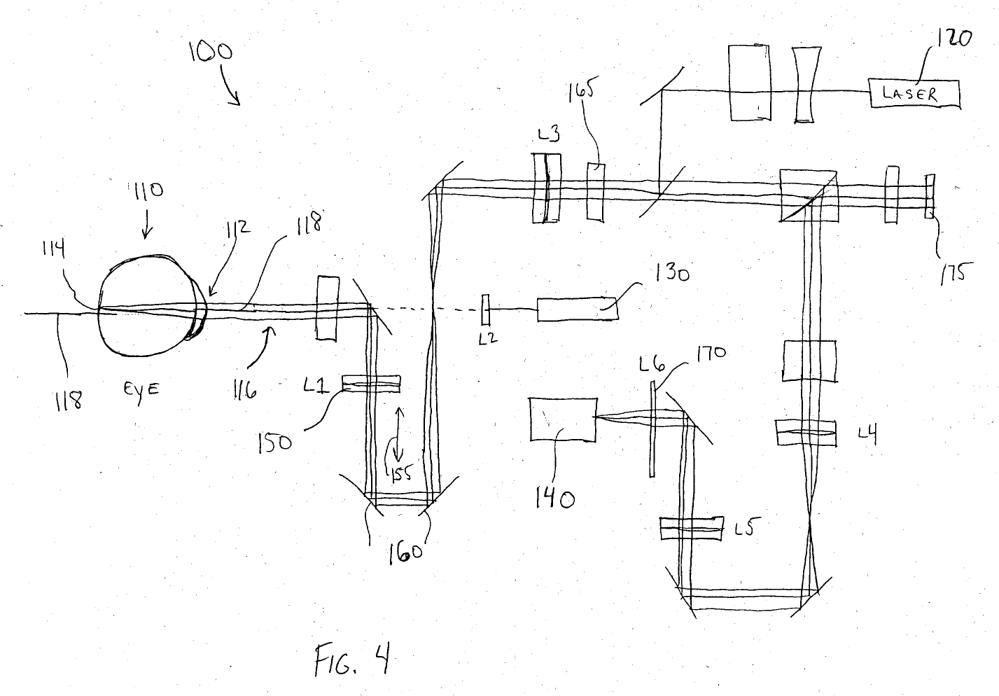

Turning now to FIG. 4, another system 100 of the present invention will be described. As discussed in conjunction with FIG. 3, system 100 may be used for imaging an eye 110 having a retina and may be operated in a similar manner as system 30. System 100 projects a light 116 or other image into and through optical tissues 112 of eye 110. Light 116 forms an image upon a surface 114 of the retina. Preferably, light 116 is transmitted into eye 110 along an optical axis 118. The proper alignment between light 116 and optical tissues 112 may be facilitated by a pupil camera 130. Similar to pupil camera 52, camera 130 images the pupil to determine a position of the pupil for registration of the wavefront sensor data relative to the eye's optical tissues 112. The light or image source may include a laser 120, a bulb or directed light in the visible range, or some other illumination mechanism.

As shown schematically in FIG. 4, system 100 includes a series of lenses and reflectors to direct the light or image into alignment with optical axis 118 and to or through various system 100 components. In one embodiment, the lenses include a lens 150 (L1) which is adapted to travel relative to reflectors 160 as shown by an arrow 155. Lens 150 is adapted to compensate for large spherical aberrations of eye 110. Further, system 100 may include an astigmatic lens 165 adapted to compensate for large irregularities in eye 110, such as astigmatism or other aberrations.

In a particular embodiment light 116 exiting anteriorly from eye 110 is directed to a lens 170 (L6) having a plurality of spaced-apart apertures. Lens 170 may be similar to, for example, lens array 38 shown in FIG. 3. Light 116 passes through lens 170 and is received by an imaging sensor 140, which in one embodiment is a Hartmann-Shack imaging sensor. Characteristics of light 116 imaged by sensor 140 can be used to determine the characteristics of an associated region of optical tissues 112.

In one embodiment, sensor 140 includes a CCD camera. In a particular embodiment, the CCD camera has a dynamic range of about 69 dB. In one embodiment, light 116 transmitted anteriorly from eye 110, through system 100 to imaging sensor 140 produces a series of bright lights or spots against a generally dark background. Further, in situations where eye 110 may contain early cataracts, the cataracts cause the reduction in the amount of light 116 passing through eye 110. This has the effect of dimming the central portion of the wavefront pattern,

During operation of the embodiment where sensor 140 is a CCD camera, the gain control of the CCD camera is set at a fixed level where the majority of data is expected. However, pixels of the CCD camera have difficulty distinguishing the high contrast images, and “blooming” may occur. Blooming involves the bleeding of pixel data into adjacent pixels, so that the data obtained by those pixels is lost or corrupted. Further, the Hartmann-Shack pattern produced by imaging a patient's eye also may have a very bright spot generated by the corneal reflex reflection. This bright spot may result in the CCD camera saturating, and the wavefront pattern being lost.

CCD cameras are particularly useful for imaging or measuring optics having a predictable or expected brightness range, and particularly for those optics having high transmission efficiencies. However, the human eye produces spots of various intensity and high contrast. Further, the light intensity or brightness received can vary from patient-to-patient and from eye-to-eye. As a result, the CCD camera dynamic range may be insufficient for accurately imaging these high contrast images.

As a result of some of these limitations, the inventor sought an improved imaging device 140. Typically, improvements in imaging involve increasing sensor resolution, and/or increasing the sensitivity of the sensor (i.e., the ability to sense darker images). However, the inventor discovered that using a CMOS imaging sensor produced exemplary results notwithstanding the fact that such a sensor typically is less sensitive and has lower resolution than a CCD camera. For example, the CMOS camera places the electronics on the imaging CMOS chip, resulting in some loss to the imaging area. The electronics may include a processor placed around or adjacent each pixel to perform logarithmic signal compression directly at the place of signal generation. The inventor determined that the desired data resides in the bright spots obtained from light or image 116. As a result, a loss in sensitivity of sensor 140, and the attendant loss in ability to sense dimmer images, would be an acceptable tradeoff to achieve increased dynamic range for imaging sensor 140. The increased dynamic range permits proper imaging of high contrast images. Imaging sensor 140 comprising a CMOS camera sufficiently captures the desired data from the bright spots generated by the wavefront.

In one embodiment of system 100, imaging sensor 140 comprises a CMOS imaging camera 140. In a particular embodiment, imaging sensor 140 comprises a HDRC CCTV camera, commercially available from IMS Chips, Inc., located in Stuttgart, Germany. It will be appreciated by those skilled in the art that other CMOS imaging devices also may be used within the scope of the present invention. In a particular embodiment, imaging sensor 140 has a dynamic range of at least about 100 decibels (dB). In another embodiment, the imaging sensor 140 has a dynamic range of at least about 120 dB. Such an increased dynamic range produces readable images, reduces or eliminates blooming, and the like.

In one embodiment, system 100 further includes a processor (not shown in FIG. 4), which may be similar to or contained in computer 22 (see FIG. 3). The processor is adapted to calculate a tomographic wavefront error map of the eye to help identify at least one aberration of the eye, calculate a treatment profile such as an ablation treatment to correct refraction, and/or to control laser 120 to illuminate and/or treat eye 110, and the like.

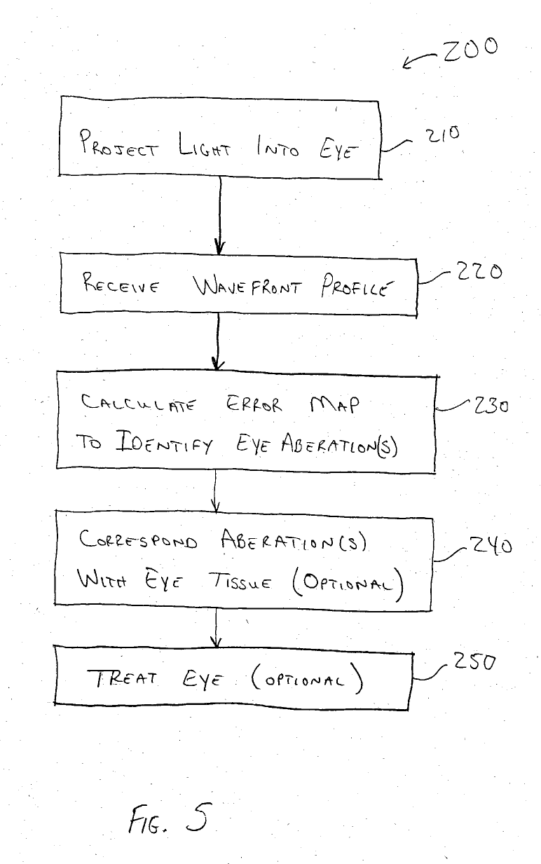

Turning now to FIG. 5, a method 200 of imaging an eye according to the present invention will be described. Method 200 includes projecting light into a patient's eye (block 210). A wavefront profile is received (block 220) from the light passing out of the patient's eye. An error map is calculated to identify eye aberration(s) (block 230). As previously described, in one embodiment the wavefront profile is received using a CMOS imaging camera having a dynamic range of at least about 100 dB. Such a camera has sufficient dynamic range to capture high contrasting images, including bright spots on a dark background. Method 200 further may optionally include corresponding the aberrations with the eye tissue or structure. In this manner, the physician or user of systems 30 and/or 100 may be able to identify the physical portion of the eye to be treated. Method 200 further optionally includes treating the eye (block 250). In one embodiment, eye treatment involves laser ablation or other treatment to correct refraction, or the like.

Notwithstanding the above description, it should be recognized that many other systems, functions, methods, and combinations thereof are possible in accordance with the present invention. Thus, although the invention is described with reference to specific embodiments and figures thereof, the embodiments and figures are merely illustrative, and not limiting of the invention. Rather, the scope of the invention is to be determined solely by the appended claims.

What is claimed is:

1. A system for diagnosing an eye of a patient, the eye having a retina and optical tissues, the system comprising:
an image source arranged to direct an image posteriorly through the optical tissues and onto the retina;
a wavefront sensor oriented to sense the image as transmitted anteriorly by the optical tissue, the wavefront sensor comprising a lenslet array and a CMOS imaging device; and
a processor coupled to the wavefront sensor for processing the image.

2. The system as in claim 1 wherein the CMOS imaging device comprises a CMOS camera having a dynamic range that is at least about 100 db.

3. The system as in claim 1 wherein the CMOS imaging device comprises a CMOS camera having a dynamic range that is at least about 120 db.

4. The system as in claim 1 wherein the processor is adapted for identifying an aberration of the eye based at least in part on the processed image.

5. The system as in claim 1 wherein the wavefront sensor comprises a Hartmann-Shack sensor.

6. The system as in claim 1 wherein the CMOS imaging device comprises a plurality of pixels including a first pixel, and wherein the imaging device is adapted to prevent a blooming effect between the first pixel and at least some of the plurality of pixels adjacent the first pixel.

7. An apparatus for measuring optical aberrations of an eye, the apparatus comprising:
a light source arranged to direct a light beam along an optical axis into the optical tissues and onto the retina; and
a wavefront sensor oriented to sense a returned image from the optical tissue, the wavefront sensor comprising a lenslet array and a CMOS imaging device;
wherein the CMOS imaging device has a dynamic range that is at least about 100 db.

8. The apparatus as in claim 7 wherein the wavefront sensor is coupled to a processor adapted for processing the returned image and determining a treatment profile for the eye.

9. The apparatus as in claim 8 wherein the processor is coupled to a laser adapted for generating a photoablative laser beam suitable for removal of corneal tissue of the eye so as to correct refraction.

10. A method for generating a profile of a patient's eye, the method comprising:
projecting a light into the patient's eye and onto a retina;
receiving a wavefront profile from the eye with an imaging device, the imaging device having a dynamic range that is at least about 100 db; and
calculating a tomographic wavefront error map of the eye to identify at least one aberration of the eye.

11. The method as in claim 10 further comprising corresponding the aberration with a tissue structure of the eye for a subsequent treatment.

12. The method as in claim 10 wherein the imaging device comprises a CMOS camera.

13. The method as in claim 10 wherein the imaging device has a dynamic range that is at least about 120 db.

14. The method as in claim 10 further comprising ablating a portion of the patient's eye in a defined pattern, the defined pattern based at least in part on the tomographic wavefront error map.
